(19) United States
(12) Patent Application Publication
(10) Pub. No.: US 2025/0022588 A1
(43) Pub. Date: Jan. 16, 2025

(54) SYSTEMS AND METHODS FOR CLASSIFICATION OF ENTITIES BASED ON METRICS

(71) Applicant: U.S. News & World Report, L.P., New York, NY (US)

(72) Inventor: Bentsion R. HARDER, Bethesda, MD (US)

(21) Appl. No.: 18/771,358

(22) Filed: Jul. 12, 2024

Related U.S. Application Data
(60) Provisional application No. 63/513,315, filed on Jul. 12, 2023.

(51) Int. Cl. G16H 40/20

(52) U.S. Cl. CPC G16H 40/20

(57) ABSTRACT

Systems and methods are disclosed for dynamically ranking entities based on comprehensive quality assessment. The method includes collecting a plurality of data from source(s); selecting, based on the plurality of data, procedure(s) and/or condition(s) upon which performance evaluation of entities is based; selecting, based on the plurality of data, entities for which the performance evaluation is conducted; for each of procedure(s) and/or condition(s): determining, using model(s), a performance score for each of the selected entities based on performance indicator(s), wherein the performance indicator(s) include one or more of: risk-adjusted outcome(s), process measure(s), and structural measure(s); and generating a rank for each of the selected entities based on the performance score determined for each of the selected entities; and causing a display of the rank for the selected entities in association with the procedure(s) and/or condition(s) in a user interface of a device.

### CROSS-REFERENCE TO RELATED APPLICATION

This application is a non-provisional of, and claims the benefit of priority to U.S. Provisional Application No. 63/513,315, filed on Jul. 12, 2023, the disclosure of which is incorporated herein in its entirety by reference.

### TECHNICAL FIELD

This present disclosure relates generally to the field of data processing and advanced analytics. In particular, the present disclosure relates to analyzing data utilizing machine-learning methodologies for ranking a plurality of entities.

### BACKGROUND

Ranking entities (e.g., hospitals) may play a crucial role in providing transparency and aiding informed decision-making for patients, healthcare providers, and policymakers alike. However, current methodologies often face significant technical challenges as they oversimplify complex healthcare metrics or rely on limited datasets that fail to capture the full spectrum of the performance of these entities. For example, existing approaches may depend on simplistic metrics which may not adequately capture the complexities of healthcare quality. These metrics often suffer from biases, inadequate risk adjustment, and incomplete datasets, leading to skewed rankings that fail to provide a comprehensive picture of an entity's performance. Moreover, traditional methods typically lack robust methods for integrating and analyzing diverse data sources, such as real-time clinical outcomes, operational efficiency metrics, and patient-reported outcomes. This fragmented approach hinders the ability to accurately assess the entity's quality across multiple dimensions and can obscure meaningful differences in care delivery. There is a pressing need for a new approach that leverages advanced statistical models, machine-learning algorithms, and big data analytics to handle the multidimensional natures of healthcare quality assessment.

### SUMMARY OF THE DISCLOSURE

The present disclosure solves the technical challenges typically encountered during the use of a conventional method, such as those discussed above. Specifically, the present disclosure solved the technical challenges by training a machine-learning model to rank one or more entities.

In some embodiments, a computer-implemented method includes: collecting, using one or more processors, a plurality of data from one or more sources; selecting, using the one or more processors and based on the plurality of data, one or more procedures and/or conditions upon which performance evaluation of one or more entities is based; selecting, using the one or more processors and based on the plurality of data, one or more entities for which the performance evaluation is conducted; for each of one or more procedures and/or conditions: determining, using the one or more processors and using one or more models, a performance score for each of the one or more selected entities based on one or more performance indicators, wherein the one or more performance indicators include one or more of: one or more risk-adjusted outcomes, one or more process measures, and one or more structural measures; and generating, using the one or more processors, a rank for each of the one or more selected entities based on the performance score determined for each of the one or more selected entities; and causing, using the one or more processors, a display of the rank for the one or more selected entities in association with the one or more procedures and/or conditions in a user interface of a device.

In some embodiments, a system for one or more processors of a computing system; and at least one non-transitory computer readable medium storing instructions which, when executed by the one or more processors, cause the one or more processors to perform operations including: collecting a plurality of data from one or more sources; selecting, based on the plurality of data, one or more procedures and/or conditions upon which performance evaluation of one or more entities is based; selecting, based on the plurality of data, one or more entities for which the performance evaluation is conducted; for each of one or more procedures and/or conditions: determining, using one or more models, a performance score for each of the one or more selected entities based on one or more performance indicators, wherein the one or more performance indicators include one or more of: one or more risk-adjusted outcomes, one or more process measures, and one or more structural measures; and generating a rank for each of the one or more selected entities based on the performance score determined for each of the one or more selected entities; and causing a display of the rank for the one or more selected entities in association with the one or more procedures and/or conditions in a user interface of a device.

In some embodiments, a non-transitory computer readable medium storing instructions which, when executed by one or more processors of a computing system, cause the one or more processors to perform operations including: collecting a plurality of data from one or more sources; selecting, based on the plurality of data, one or more procedures and/or conditions upon which performance evaluation of one or more entities is based; selecting, based on the plurality of data, one or more entities for which the performance evaluation is conducted; for each of one or more procedures and/or conditions: determining, using one or more models, a performance score for each of the one or more selected entities based on one or more performance indicators, wherein the one or more performance indicators include one or more of: one or more risk-adjusted outcomes, one or more process measures, and one or more structural measures; and generating a rank for each of the one or more selected entities based on the performance score determined for each of the one or more selected entities; and causing a display of the rank for the one or more selected entities in association with the one or more procedures and/or conditions in a user interface of a device.

It is to be understood that both the foregoing general description and the following detailed description are example and explanatory only and are not restrictive of the detailed embodiments, as claimed.

### BRIEF DESCRIPTION OF THE DRAWINGS

The accompanying drawings, which are incorporated in and constitute a part of this specification, illustrate various example embodiments and together with the description, serve to explain the principles of the disclosed embodiments.

FIG. 1 is a diagram showing an example of a system for dynamically ranking one or more entities based on comprehensive quality assessment, according to aspects of the disclosure.

FIG. 2A is a flowchart of a process for generating a rank based on the performance evaluation of one or more entities on selected procedure(s) and/or condition(s), according to aspects of the disclosure.

FIG. 2B is a flowchart of a process for dynamically ranking one or more entities by leveraging comprehensive quality assessment, according to aspects of the disclosure.

FIG. 3 illustrates an acyclic graph that shows the hypothesized relationship between covariates, hospital selection, and outcomes, according to aspects of the disclosure.

FIG. 4 is a flowchart of a process for determining whether an entity is recognized as the best service provider, according to aspects of the disclosure.

FIG. 5 is a flowchart of a process for a scoring methodology that determines the state and metro area rankings of entities, according to aspects of the disclosure.

FIG. 6 shows an example machine-learning training flow chart.

FIG. 7 illustrates an implementation of a computer system that executes techniques presented herein.

### DETAILED DESCRIPTION OF EMBODIMENTS

This present disclosure relates generally to the field of data processing and advanced analytics. In particular, the present disclosure relates to analyzing data utilizing machine-learning methodologies for ranking a plurality of entities.

Ranking entities (e.g., hospitals) may present significant technical challenges due to the inherent complexity of healthcare data and the multifaceted nature of an entity's performance. One issue is the integration and standardization of diverse data sources, including clinical outcomes, patient demographics, operational metrics, and patient-reported experiences. These datasets often vary in format, granularity, and reliability, making it difficult to create a unified and comprehensive ranking system. Moreover, existing methodologies frequently rely on simplistic or unweighted aggregation of these metrics, which can obscure important nuances and lead to inaccurate assessments of the entity's performance. The variability in data quality and completeness further exacerbates these issues, requiring sophisticated data preprocessing and normalization techniques to ensure fairness and comparability.

Conventional methodologies are often technically deficient due to their reliance on limited and static datasets. Many traditional systems prioritize easily quantifiable metrics without adequately accounting for context or underlying factors that may influence those outcomes. For example, hospitals serving high-risk populations or those with complex medical needs may be unfairly penalized despite providing high-quality care. Current methods also frequently lack robust risk adjustment mechanisms to account for patient severity and comorbidities, leading to skewed rankings that do not reflect true performance differences. Additionally, the absence of real-time data integration limits the ability of these systems to provide up-to-date and relevant insights, further diminishing their utility for stakeholders seeking accurate and actionable information.

Current methodologies are limited in their technical capacity to handle and process the vast volumes of unstructured data generated in healthcare settings. Traditional systems often rely primarily on structured data, such as numerical and categorical data from electronic health records (EHRs), and may ignore valuable unstructured data sources like clinical notes, imaging reports, and patient feedback. These unstructured data contain rich and nuanced information that can provide deeper insight into hospital performance. The inability to integrate and analyze unstructured data may lead to incomplete and potentially biased rankings. This technical deficiency underscores the need for more sophisticated data processing capabilities that can leverage the full spectrum of available healthcare data, ensuring a more accurate and holistic assessment of hospital quality.

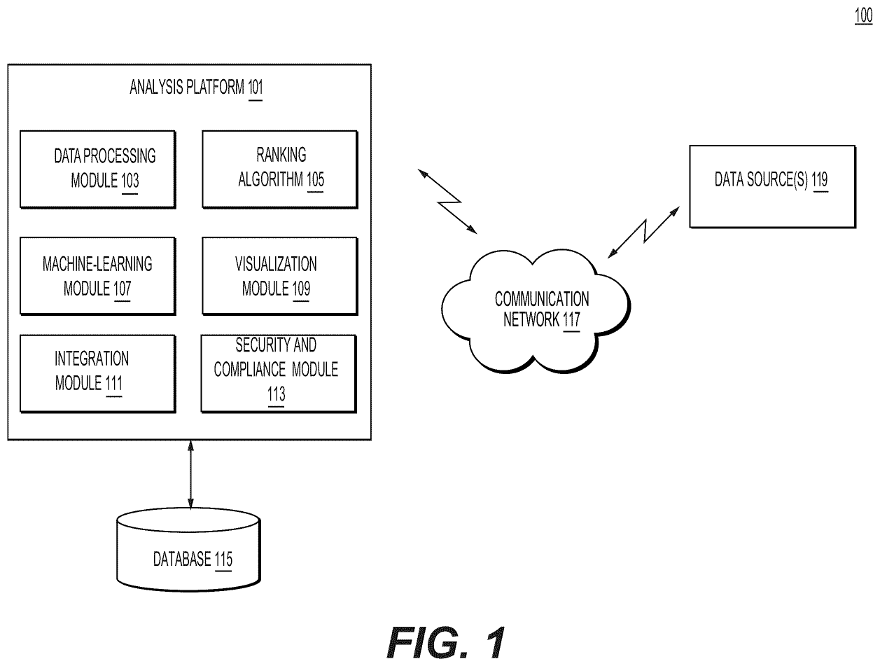

System 100 of FIG. 1 may address the limitations of the conventional methods through a multifaceted approach that leverages advanced data processing, machine-learning, and sophisticated statistical techniques. Unlike the traditional methodologies that often rely on static models and limited data points, the system 100 may dynamically incorporate a vast array of data sources, including publicly available indicators, specialized datasets (e.g., Medicare Beneficiary Summary File (MBSF), Medicare inpatient Limited Data Sets Standard Analytical Files (LDS SAF), and American Hospital Association (AHA) surveys, and detailed clinical outcomes. By utilizing comprehensive risk adjustment variables (e.g., age, sex, comorbidities, and socioeconomic status) the system 100 may ensure a more accurate comparison of hospitals, effectively normalizing for patient demographics and health conditions. In one example, system 100 may rank hospitals by assigning points based on their performance across various procedures and/or conditions, deducting points for below-average ratings, and ultimately ranking hospitals regionally and metro-wise. This process may reduce biases inherent in the conventional methods and may provide a more equitable assessment of hospital performances.

Furthermore, the system 100 may employ advanced ranking algorithms and machine-learning models to determine the optimal weighting of quality indicators, addressing the issues in constructing composite ratings. These algorithms may empirically derive the significance of each indicator based on its predictive value and may adjust for measurement errors due to incomplete risk adjustments or random variation from low sample sizes. System 100 may implement rigorous inclusion and exclusion criteria that may ensure that only hospitals meeting high standards in specific procedures and/or conditions are considered for ranking. By continuously updating and refining the models with new data, the system 100 may maintain relevance and accuracy, unlike status models that quickly become outdated. The inclusion of process and structural measures, alongside outcome-based metrics, may offer a holistic view of hospital quality, encompassing factors like nurse staffing ratios, compliance with treatment protocols, and accreditation status. This comprehensive and adaptive approach may ensure that the rankings are not only more precise but also more reflective of actual hospital performance.

FIG. 1 is a diagram showing an example of a system for dynamically ranking one or more entities based on comprehensive quality assessment, according to aspects of the disclosure. FIG. 1 includes the system 100 that comprises analysis platform 101, a database 115, a communication network 117, and data source(s) 119.

In one embodiment, the analysis platform 101 is a platform with multiple interconnected components. The analysis platform 101 includes one or more servers, intelligent networking devices, computing devices, components, and corresponding software for dynamically ranking one or more entities based on comprehensive quality assessment using integrated data sources, advanced risk adjustment, and machine-learning techniques. In one instance, the analysis platform 101 may collect extensive data from various sources, including publicly available indicators, specialized datasets, and clinical outcomes, to evaluate the performance of entities like hospitals across multiple procedures and/or conditions. The analysis platform 101 may apply criteria to assign or deduct points based on their performance metrics within these areas. The analysis platform 101 may aggregate the points and rank hospitals to provide a comprehensive assessment of their healthcare quality. The rankings may be displayed in a user-friendly interface on a device, facilitating transparent and accessible comparisons. The functions and components of the analysis platform 101 are discussed in detail throughout the disclosure.

In one instance, the analysis platform 101 may include a data processing module 103, a ranking algorithm 105, a machine-learning module 107, a visualization module 109, an integration module 111, a security and compliance module 113, or any combination thereof. As used herein, terms such as “component” or “module” generally encompass hardware and/or software, e.g., that a processor or the like used to implement associated functionality. It is contemplated that the functions of these components are combined in one or more components or performed by other components of equivalent functionality.

In one instance, the data processing module 103 may ingest raw data from various sources such as data source(s) 119, EHRs, patient satisfaction surveys, hospital administrative databases, and external public health databases. The raw data may be in diverse formats and structures, necessitating extract, transform, load (ETL) processes to ensure consistency and reliability. The data processing module 103 may perform data cleaning on the raw data, by identifying and rectifying anomalies, such as missing values, duplicates, and outliers using sophisticated algorithms. The data processing module 103 may implement transformation processes to convert the data into a standardized format, employing techniques like data parsing and encoding to ensure interoperability between different data sources. The data processing module 103 may normalize the data to ensure that disparate data metrics are scaled to a common range, thereby allowing fair comparisons. Advanced techniques like natural language processing (NLP) may be employed to extract meaningful information from unstructured data sources, such as physician's notes and patient feedback.

In one instance, ranking algorithms 105 may systematically analyze data from various sources, including patient outcomes, healthcare quality metrics, and procedural success rates, to generate comprehensive rankings. The ranking algorithm 105 may measure the performance of hospitals across, for example, 21 common inpatient procedures and conditions by systematically evaluating specific clinical outcomes and quality metrics for each. The 21 areas assessed may include:
1. Abdominal aortic aneurysm repair (AAA);
2. Aortic valve surgery (AVR);
3. Chronic obstructive pulmonary disease (COPD);
4. Colon cancer surgery;
5. Heart failure (CHF);
6. Diabetes;
7. Back surgery (Spinal fusion);
8. Heart attack;
9. Coronary Artery Bypass Grafting (CABG);
10. Hip fracture;
11. Hip replacement;
12. Kidney failure;
13. Knee replacement;
14. Leukemia, lymphoma & myeloma;
15. Lung cancer surgery;
16. Ovarian cancer surgery;
17. Pneumonia;
18. Prostate cancer surgery;
19. Stroke;
20. Transcatheter aortic valve replacement (TAVR); and
21. Uterine cancer surgery.

The ranking algorithm 105 may analyze data such as patient mortality rates, complication rates, readmission rates, and patient satisfaction scores for each procedure and condition. Hospitals may earn points based on their performance, with higher points awarded for exceptional outcomes and penalization for below-average results. By integrating these data points, the ranking algorithm 105 may provide a comprehensive and objective ranking of hospitals, highlighting those that consistently deliver high-quality care across a broad spectrum of medical and surgical services. In one example, procedures such as AAA, AVR, and COPD treatments may be evaluated based on factors like mortality rates, complication rate, and adherence to clinical guidelines. In one example, conditions like colon cancer surgery, CHF, diabetes, and back surgery (Spinal fusion) outcomes may be assessed for their effectiveness in patient care and recovery. Specialized procedures such as hip and knee replacements, treatments for Leukemia, lung cancer, ovarian cancer, and prostate cancer surgeries may be rated based on surgical outcomes, patient recovery, and long-term health outcomes.

In one instance, the ranking algorithm 105 may select the procedures and conditions based on three key criteria: (i) frequency of admissions in the Medicare population, (ii) the ability to make standardized hospital-to-hospital comparisons, and (iii) the presence of sufficient risk or complexity. This may ensure that the rankings focus on commonly encountered and highly relevant areas of care, enable fair and accurate benchmarking across hospitals, and highlight the importance of quality performance in complex or high-risk procedures. By adhering to these criteria, the ranking algorithm 105 may provide meaningful and actionable insights into hospital performances, guiding patients in their healthcare decisions and promoting improvements in care quality.

In one instance, the ranking algorithm 105 may define inclusion and exclusion criteria for maximizing statistical and clinical accuracy by considering:
1. Maximal homogeneity: This ensures that the patient groups being compared are as similar as possible, reducing variability and enhancing the reliability of comparisons. For example, patients are as alike as possible other than with regard to factors that could be adequately managed through risk adjustment.
2. Maximal sample size: This ensures that there are enough data points to achieve robust statistical power and meaningful results. For example, the selection of procedure and condition cohorts is limited to those with a sufficiently large volume of care for statistical robustness and meaningfulness.
3. Minimal coding variation: This may reduce discrepancies in recording medical conditions and procedures, ensuring that comparisons across hospitals are accurate and consistent. For example, coding definitions are relatively immune to large variations due to differences in coding practices. In considering this issue, it is particularly important to try to avoid systematic biases that may benefit particular organizations and encourage gaming, as opposed to random coding variations that would simply add noise and reduce precision.

In one instance, ranking algorithms 105 may also incorporate risk adjustment methodologies to account for patient demographics, comorbidities, and other variables that may influence outcomes. When comparing outcomes between hospitals, adjusting for differences in the patients treated at each hospital is critical. For example, a hospital with a 50% mortality rate might be superior to a hospital with a 10% mortality rate if most of the patients at the first hospital are expected to die and most of the patients at the second hospital are low risk. A multilevel logistic regression model may be utilized to adjust for differences in case mix between hospitals. Multilevel models may be a form of regression that allocates variance between variables on two or more levels. In one example, an empirical Bayes estimate of the hospital intercept may be used as an estimate of each hospital's value for a given outcome. Multilevel modeling may account for the clustering of patient observations within hospitals and may allow for a more precise rating of hospitals with lower patient volume and fewer outcomes.

By analyzing a broad range of indicators and conditions, ranking algorithms may provide a comprehensive assessment of a hospital's performance, enabling stakeholders to make informed decisions about care quality and provider selection across diverse medical specialties. For example, the ranking algorithm 105 may take into consideration the following as risk adjustment variables:
1. Age at admission. Age is a critical factor in assessing patient risk and outcomes, as older patients often have a higher risk of complications and mortality. Adjusting for age ensures that hospitals treating an older population are fairly compared to those with younger patients.
2. Sex: Biological differences between males and females can influence health outcomes and responses to treatments. Adjusting for sex may help to account for these differences, ensuring that comparisons between hospitals are not biased by gender composition.
3. Inbound transfer status: Patients transferred from another hospital (inbound transfer) are often more complex and critically ill. Accounting for transfer status may ensure that hospitals receiving these higher-risk patients are not unfairly penalized in the rankings.
4. Year of hospital admission: Medical practices, technologies, and standards of care may evolve over time. Adjusting for the year of admission may help to account for these temporal changes, ensuring that comparisons reflect current standards and do not unfairly benefit or disadvantage hospitals based on when they treat patients.
5. Elixhauser comorbidities: The Elixhauser comorbidities index may include a comprehensive set of comorbid conditions that may affect patient outcomes. Adjusting for these comorbidities may provide a more accurate assessment of hospital performance by considering the overall health and complexity of the patients they treat.
6. Medicare Status Code: The Medicare Status Code may provide additional information about the patient's eligibility and coverage, which may influence their access to care and overall health outcomes. Adjusting for Medicare status may help to ensure that hospitals are compared fairly, regardless of the specific patient populations they serve.
7. Socioeconomic status: Socioeconomic factors, such as income, education, and housing may significantly impact health outcomes. For example, patients with lower incomes are typically sicker when they arrive at the hospital, and may face more challenges in obtaining or managing their care after they are discharged. This may affect their risk of death, readmission, and complications. When hospitals differ by the socioeconomic status of their patients, this can create bias in comparing outcomes. Adjusting for socioeconomic status may help to account for these external factors, ensuring that hospitals serving disadvantaged populations are not unfairly penalized.
8. Condition cohort-specific covariates: Specific conditions may have unique risk factors that may need to be considered. For example, binary variables indicating whether a patient had ever left against medical advice, been admitted for the same condition, or had a history of mechanical ventilation may be included in CHF and COPD models. For example, respiratory failure may be risk-adjusted in COPD outcomes models. For example, a binary measure indicating whether a patient was diagnosed with acute leukemia is included in the leukemia, lymphoma & myeloma model. For example, a binary measure indicating whether a patient had a diagnosis of sepsis is included in the pneumonia cohort. For example, binary variables indicating whether a patient had a diagnosis of ST-elevation myocardial infarction (STEMI) of the anterior wall, STEMI of the inferior wall, or non-ST elevation myocardial infarction (NSTEMI) are included in the heart attack models. For example, binary variables indicating whether a patient was diagnosed with diabetes ketoacidosis (DKA) and hypoglycemia are included in the diabetes models. Adjusting for these condition-specific covariates may ensure that the rankings accurately reflect the hospital's performance for each specific procedure or condition.
9. Surgical cohort-specific covariates: Variables or factors that are specific to particular groups or categories of surgical patients and influence outcomes and treatment approaches. A binary variable indicating whether the operation was performed on both joints simultaneously (bilaterally) may be included in the hip replacement and knee replacement models. A binary variable indicating approach (open or endoscopic) may be included in the AAA mortality model. A binary variable indicating diagnosis of CHF or heart attack may be included in the CABG models. An ordinal variable indicating the type of degenerative condition (e.g., scoliosis) may be included in the back surgery models. A binary variable indicating whether a patient also had a secondary diagnosis of the other cancer may be included in the ovarian and uterine models.
10. History of Stroke: A variable indicating history of stroke in the year prior to surgery may be included in the stroke model.
11. Covid-19 diagnosis. Patients diagnosed with Covid-19 in 2021 (and onward) are risk-adjusted in all procedure and condition outcomes models.

In one instance, evaluation of the risk-adjustment model may involve assessing its effectiveness in accounting for variations in patient demographics, clinical characteristics, and other factors that influence health outcomes. Key aspects of evaluating a risk adjustment model may include:
1. C-statistic: The C-statistic may estimate the probability that if one subject who experienced an outcome (death, for example) and another who did not are drawn randomly from the data, the model will assign a higher probability of death to the person who died. A C-statistic of 0.5 may indicate the model has no better than random chance at predicting the outcome. A C-statistic in the 0.60-0.69 range may indicate limited discrimination, 0.70-0.79 may indicate reasonable discrimination, and above 0.8 may indicate good discrimination.
2. Hosmer-Lemeshow goodness of fit statistic: This statistic may look at whether the observed number of outcomes matches the expected number predicted by the model in samples of the population. A low p-value in the Hosmer-Lemeshow test may suggest a poor fit of the model, indicating discrepancies between predicted and actual outcomes that may require adjustment in the model's specification or data used.
3. Predictive accuracy: Evaluating how well the model predicts outcomes compared to actual observed outcomes.
4. Calibration: Assessing whether the predicted probabilities of outcomes align closely with the actual observed probabilities across different patient subgroups.
5. Generalizability: Determining how well the risk adjustment model performs across different populations, healthcare settings, and time periods.
6. Bias and fairness: Examining whether the risk adjustment model introduces bias or unfairness in healthcare decision-making, particularly in terms of race, ethnicity, socioeconomic status, or other demographic factors.

In one instance, in developing comprehensive ranking algorithm 105, the analysis platform 101 may evaluate a variety of process measures and inpatient datasets to ensure accuracy and reliability. Many potential measures may be excluded due to issues such as missing data, which may compromise the integrity of the analysis or other concerns about data validity. Additionally, some measures may not demonstrate a good empirical fit, meaning they do not reliably predict outcomes or show significant variability among hospitals. In one example, based on rigorous evaluation, the analysis platform 101 may include the following measures:
1. Worker flu immunization: This measure may reflect the percentage of healthcare workers who may receive timely vaccination during flu season. High immunization rates among staff reduce the spread of flu within hospitals, protecting both the patients and employees. This is an important indicator of hospital efforts to prevent infections and promote a safe environment.
2. Noninvasive ventilation: The use of noninvasive ventilation (NIV) may be a critical process measure for patients with respiratory failure COPD. Proper implementation of NIV may improve patient outcomes and may reduce the need for invasive procedures, making it a valuable measure of respiratory care quality.
3. Patient experience: Patient experience scores (e.g., linear mean score of recently discharged patient experience) may reflect perceptions of their care, including communication with healthcare providers, the responsiveness of hospital staff, and overall satisfaction. These scores may be crucial for assessing the quality of patient-centered care and the hospital's ability to meet patient needs and expectations. In one example, for back surgery (spinal fusion), hip fracture, hip replacement, and knee replacement cohorts, an adjustment may be introduced to account for the fact that Hospital Consumer Assessment of Healthcare Providers and Systems (HCAHPS) scores tend to be higher at specialty hospitals versus general acute-care hospitals. This may be due to different characteristics in the patient population and not wholly the result of different outcomes. The group mean adjustment may bring the mean HCAHPS scores at specialty hospitals closer to the mean scores at general hospitals to ensure that scores are comparable across hospital service categories.
4. Board certification: The percentage of physicians who are board-certified in their specialties indicates the level of expertise and qualification among the hospital's medical staff. Board certification is associated with higher standards of practice and better patient outcomes, making it a key measure of medical quality.
5. Emergency room visits after chemotherapy: This measure may track the frequency of unplanned emergency room visits following chemotherapy treatment. High rates may indicate issues with managing chemotherapy side effects or complications, highlighting areas for improvement in oncology care.
6. Unplanned visits after colonoscopy: Similar to the chemotherapy measure, this tracks the rate of unplanned visits following colonoscopy procedures. High rates may signal complications or inadequate pre-procedure assessment, serving as an important indicator for gastrointestinal care.
7. Compliance with the septic shock bundle: Compliance with the septic shock bundle may measure adherence to evidence-based protocols for managing septic shock. High compliance rates are associated with improved patient outcomes in sepsis care, making this a critical measure of a hospital's performance in managing severe infections.
8. Public transparency: This measure may track recognized hospitals that are publicly acknowledged for their quality of care by reputable organizations. These may include:
   (i) Get With The Guideline (GWTG) recognized hospital: This recognition indicates adherence to American Heart Association guidelines for cardiovascular care;
   (ii) American College of Cardiology (ACC) recognized hospital: This recognition reflects excellence in cardiology care based on ACC standards;
   (iii) Society of Thoracic Surgeons (STS) recognized hospital: This acknowledgment recognizes high-quality thoracic surgery programs;
   (iv) STS/ACC TVT registry recognized hospital: This indicates participation in the transcatheter valve therapy registry, reflecting high standards in valve replacement procedures.

In one instance, the ranking algorithm 105 may implement a structural measure of healthcare to assess the attributes of the setting in which care is provided. These measures may be crucial because they reflect the capacity of healthcare facilities to deliver high-quality care. Structural indicators that have been empirically associated with good patient outcomes are included in the ranking. Specifically, the following structural indicator may be employed to provide a comprehensive evaluation:
1. Volume: It may refer to the number of specific procedures or treatments performed by a hospital. Higher volumes are often associated with better outcomes due to the increased experience and proficiency of the healthcare providers. Hospitals with higher volumes of certain procedures may have more specialized staff and resources, contributing to improved patient care.
2. Nurse staffing: The number of nurses involved in direct patient care at a hospital is known to play a major role in the quality of care. Nurse staffing levels are a critical structural measure, as adequate nurse staffing is linked to better patient outcomes, including lower rates of complications, infections, and mortality. This measure may evaluate the number of full-time equivalent (FTE) registered nurses available to care for the patients.
3. Nurse staffing index: This is a specific metric that may calculate the ratio of FTE registered nurses to adjusted patient days. This ratio may provide a standardized measure of nurse availability relative to patient volume, offering insight into the hospital's capacity to provide adequate nursing care. Higher ratios may indicate better nurse staffing levels, which are associated with improved patient outcomes.
4. National Cancer Institute (NCI) designated Cancer Center and/or American College of Surgeons (ACS) Commission on Cancer: Hospitals that are designated as NCI cancer centers or accredited by the ACS commission on Cancer are recognized for their excellence in cancer care. These designations indicate that the hospital meets rigorous standards for cancer treatment, research, and patient care, suggesting high-quality structural capabilities in oncology.
5. Surgical cohort-specific covariates: This measure includes specific structural indicators relevant to surgical cohorts, such as the availability of specialized surgical teams, advanced surgical equipment, and adherence to surgical protocols. These covariates may ensure that the structural capacity for high-quality surgical care is accurately assessed, contributing to better surgical outcomes.

The ranking algorithm 105 by incorporating these structural indicators may ensure that the evaluated hospitals have the necessary infrastructure to provide high-quality care. These measures may reflect the hospital's capability to deliver effective treatments, manage complex cases, and maintain a high standard of patient care, ultimately contributing to better health outcomes.

In one instance, the ranking algorithm 105 may construct composite rating of the quality of surgical or medical care by:
1. Determining the appropriate weight for each quality indicator. The ranking algorithm 105 may establish how much weight each quality indicator should receive in the composite rating. Each indicator, such as mortality rates, complication rates, patient satisfaction, or adherence to clinical guidelines, may provide unique insights into the quality of care. However, these indicators may vary in their importance and relevance to different aspects of care. Assigning appropriate weights may ensure that the composite rating accurately reflects the overall quality of care. Sophisticated statistical procedures, such as factor analysis or regression models, may empirically determine the optimal weights for each indicator. These methods may analyze the relationships between indicators and outcomes to assign weights based on their predictive value and impact on patient outcomes.
2. Accounting for measurement errors that may arise from several sources, such as:
   (i) Incomplete risk adjustment: If risk adjustment is incomplete or inadequate, it may lead to biased comparisons. Risk adjustment accounts for patient factors that influence outcomes, ensuring fair comparisons between hospitals. Incomplete adjustments may skew the result, making some hospitals appear better or worse than they are.
   (ii) Random variation due to low sample size: Smaller hospitals or those with fewer cases may exhibit greater random variation in their quality indicators. This random variation may lead to unstable or unreliable measurements, affecting the accuracy of the composite rating.
   (iii) Other factors: data entry errors, variations in coding practices, or differences in patient populations, may also contribute to measurement errors.

In one instance, the ranking algorithm 105 may implement advanced statistical techniques, such as hierarchical modeling or Bayesian methods, to address these issues. These techniques may account for random variations and measurement errors, providing more reliable and stable estimates of hospital performance. They can also incorporate adjustments for incomplete risk adjustment, ensuring that the composite ratings accurately reflect the true quality of care provided by each hospital.

In one instance, the machine-learning module 107 may leverage advanced algorithms and models to analyze complex healthcare data and generate accurate performance rankings. The machine-learning module 107 may employ a variety of machine-learning techniques including supervised learning algorithms like regression models, decision, trees, and ensemble methods (e.g., Random Forests, Gradient Boosting Machines) that utilizes training data, e.g., training data 612 illustrated in the training flow chart 600, for training a machine-learning model configured to rank a plurality of hospitals. The machine-learning module 107 may perform model training using training data, e.g., data from other modules, that contains input and correct output, to allow the model to learn over time. The training is performed based on the deviation of a processed result from a documented result when the inputs are fed into the machine-learning model, e.g., an algorithm measures its accuracy through the loss function, adjusting until the error has been sufficiently minimized. The machine-learning module 107 may also employ unsupervised learning techniques such as clustering and dimensionality reduction.

In one example, these algorithms are trained on historical data from diverse sources, allowing the system to identify patterns, predict outcomes, and adjust for risk factors. The machine-learning module 107 may incorporate feature engineering processes to extract and transform relevant data points, enhancing the predictive power of the models. Additionally, the machine-learning module 107 may utilize techniques like cross-validation and hyperparameter tuning to optimize model performance and ensure robustness. By continuously learning from new data inputs and feedback loops, the machine-learning module 107 may adapt to evolving trends in healthcare, ensuring the hospital rankings remain accurate, reliable, and reflective of current performance metrics. This dynamic capability enables the system to provide stakeholders with up-to-date and actionable insights for informed decision-making.

In one instance, the machine-learning module 107 may leverage deep learning to process unstructured data, including medical images and free-text clinical notes, using Convolutional Neural Networks (CNNs) and Recurrent Neural Networks (RNNs). NLP may extract valuable insights from patient reviews and physicians' notes, enriching the performance evaluation. The machine-learning module 107 may utilize transfer learning for fine-tuning pre-trained models on specific hospital data, enhancing performance with reduced computational resources. The machine-learning module 107 may implement temporal analysis to capture trends and changes over time, ensuring rankings remain current and relevant, while anomaly detection algorithms identify outliers and unusual patterns, maintaining the robustness of the system.

In one instance, the visualization module 109 may transform complex data and model outputs into intuitive and actionable insights. The visualization module 109 may utilize advanced data analytics techniques to perform in-depth analysis, uncovering patterns, trends, and correlations within the healthcare data. The visualization module 109 may generate interactive dashboards and displays to allow users to explore the data dynamically, facilitating a deeper understanding of hospital performance across various metrics. In one example, key performance indicators (KPIs) may be displayed through visually engaging charts, graphs, and heatmaps, enabling stakeholders to quickly identify strengths and areas for improvement. Additionally, the visualization module 109 may support drill-down capabilities, allowing users to delve into granular data for specific procedures, conditions, or demographic groups. In one example, real-time analytics features may provide up-to-date information, reflecting the latest data inputs and model updates. By making complex data accessible and understandable, the visualization module 109 may empower healthcare providers and administrators to make informed, data-driven decisions aimed at improving hospital quality and patient outcomes.

In one instance, the integration module 111 may ensure seamless interoperability and data exchange between various healthcare systems and external data sources. The integration module 111 may employ various APIs (e.g., RESTful API) to facilitate secure, efficient, and scalable communication, allowing disparate systems to interact and share data effortlessly. Through standardized data formats such as JSON and XML, the integration module 111 may ensure compatibility and integration with EHRs, patient management systems, and public health databases. Additionally, the integration module 111 may support various authentication mechanisms to guarantee secure access to sensitive healthcare data. The integration module 111 may also include real-time data sharing capabilities, enabling continuous data flow and timely updates to the ranking system. By providing robust integration capabilities, the integration module 111 may allow for the aggregation of comprehensive data from multiple sources, ensuring that the hospital ranking system remains accurate, current, and reflective of real-world conditions.

In one instance, the security and compliance module 113 may implement advanced encryption protocols, such as AES-256 and TLS, to protect data both at rest and in transit, preventing unauthorized access and data breaches. Robust access control mechanisms, including role-based access control (RBAC) and multi-factor authentication (MFA), may ensure that only authorized personnel can access critical data and system functionalities. Additionally, the security and compliance module 113 may incorporate comprehensive auditing and logging features to monitor and track system activity, providing a detailed record of compliance verification and incident response. For example, compliance with healthcare regulations is rigorously enforced through continuous monitoring and regular security assessments. By integrating these stringent security measures and compliance practices, the security and compliance module 113 may ensure the integrity, confidentially, and availability of healthcare data.

In one instance, the database 115 may store, manage, and retrieve vast amounts of healthcare data with high efficiency and reliability. The database 115 may employ advanced database technologies, such as relational databases (e.g., MySQL) for structured data and NoSQL databases for unstructured and semi-structured data, ensuring optimal performance and scalability. The database 115 may support complex queries and transactions, allowing for real-time data analytics and rapid access to critical information. The database 115 may implement robust indexing and partitioning strategies to enhance query performance and manage large datasets effectively. Additionally, the database 115 may integrate backup and disaster recovery solutions to ensure data integrity and availability in the event of system failure. Data integrity is further maintained through constraints, triggers, and stored procedures, enforcing business rules and data validation. By leveraging these advanced database management practices, the database 115 may provide a solid foundation for the analysis platform 101, enabling it to handle the complexity and scale of healthcare data with ease.

In one instance, various elements of the system 100 may communicate with each other through the communication network 117. The communication network 117 may support a variety of different communication protocols and communication techniques. In one embodiment, the communication network 117 may allow data source(s) 119 to communicate with the analysis platform 101. The communication network 117 of the system 100 may include one or more networks such as a data network, a wireless network, a telephony network, or any combination thereof. It is contemplated that the data network is any local area network (LAN), metropolitan area network (MAN), wide area network (WAN), a public data network (e.g., the Internet), short range wireless network, or any other suitable packet-switched network, such as a commercially owned, proprietary packet-switched network, e.g., a proprietary cable or fiber-optic network, and the like, or any combination thereof. In addition, the wireless network is, for example, a cellular communication network and employs various technologies including 5G (5th Generation), 4G, 3G, 2G, Long Term Evolution (LTE), wireless fidelity (Wi-Fi), Bluetooth®, Internet Protocol (IP) data casting, satellite, mobile ad-hoc network (MANET), vehicle controller area network (CAN bus), and the like, or any combination thereof.

In one instance, data source(s) 119 may encompass a wide array of datasets essential for comprehensive performance assessment of the hospitals. These sources may include publicly available indicators, such as Medicare's Hospital Compare data, which may provide insight into hospital quality measures like mortality rates, readmission rates, and patient safety indicators. The MBSF and LDS SAF may offer detailed patient-level information crucial for risk adjustment and outcome analysis. Additionally, the AHA Annual survey may contribute data on hospital characteristics and operational metrics, while the HCAHPS survey may capture patient-reported experiences and satisfaction. In one instance, specialized datasets like those from the American Board of Orthopedic Surgery verification data may provide specific performance metrics related to orthopedic care quality. Integrating these diverse data sources may involve rigorous data governance and integration processes to ensure data accuracy, consistency, and interoperability across different formats and sources. By leveraging these comprehensive data sources, the analysis platform 101 may provide a nuanced evaluation of the hospital's performance across various dimensions, thereby facilitating information decision-making.

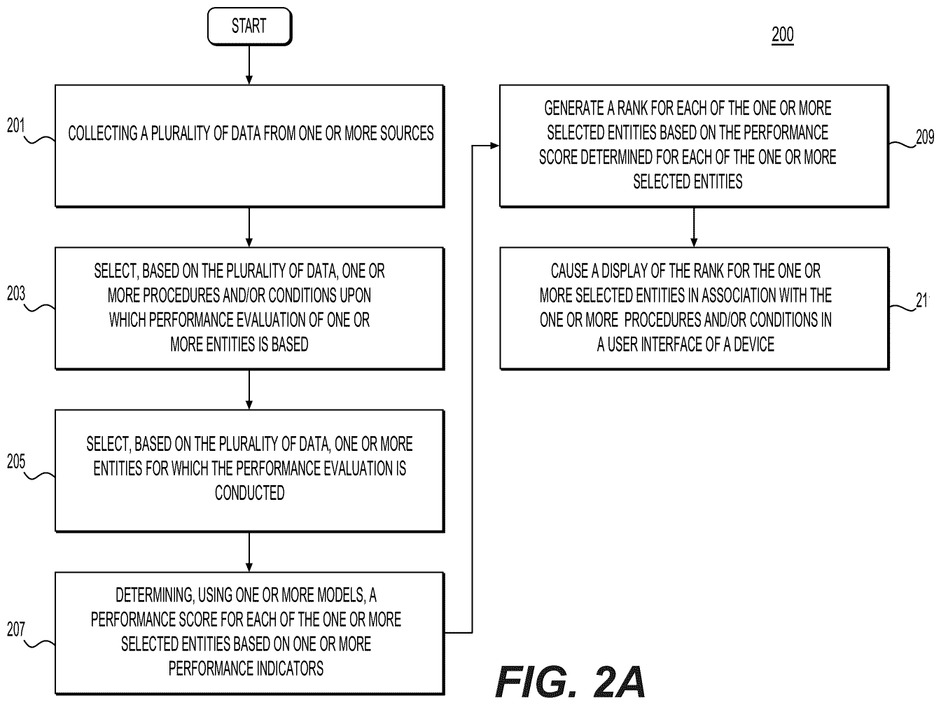

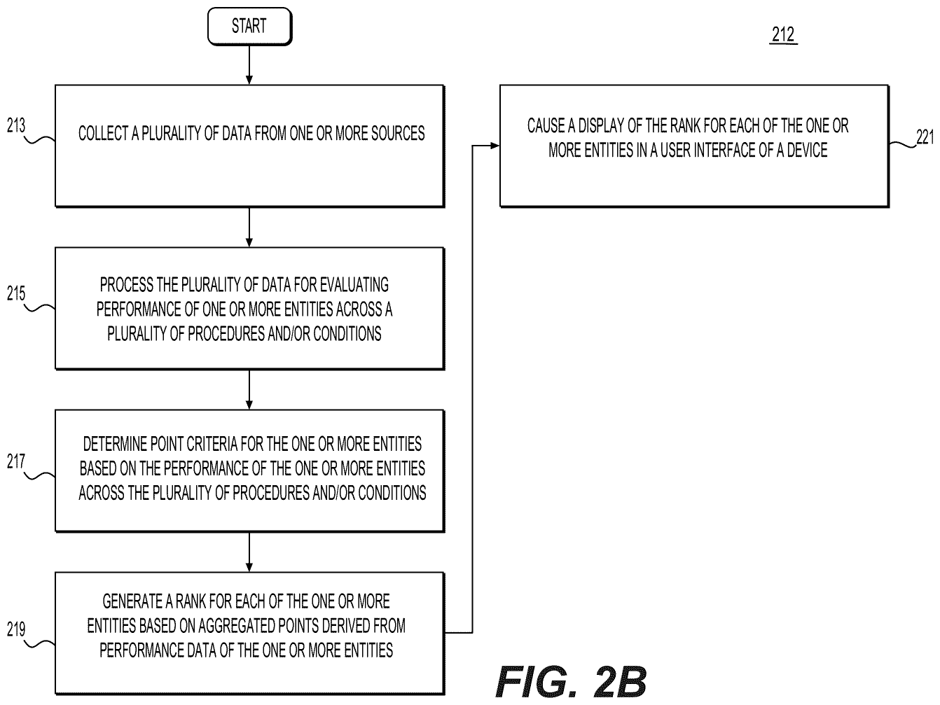

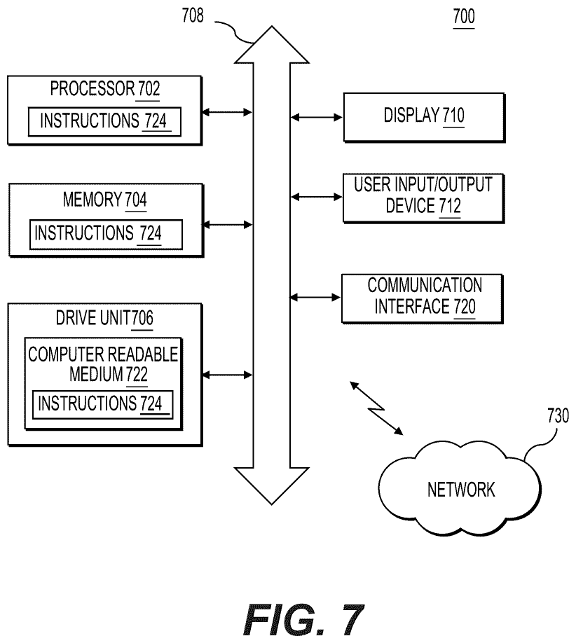

FIG. 2A is a flowchart of a process for generating a rank based on the performance evaluation of one or more entities on selected procedure(s) and/or condition(s), according to aspects of the disclosure. In various embodiments, the analysis platform 101 and/or any of the modules 103-113 may perform one or more portions of the process 200 and are implemented using, for instance, a chip set including a processor and a memory as shown in FIG. 7. As such, the analysis platform 101 and/or any of modules 103-113 may provide means for accomplishing various parts of the process 200, as well as means for accomplishing embodiments of other processes described herein in conjunction with other components of the system 100. Although the process 200 is illustrated and described as a sequence of steps, it is contemplated that various embodiments of the process 200 may be performed in any order or combination and need not include all of the illustrated steps.

In step 201, the analysis platform 101 may collect a plurality of data from one or more sources (e.g., data source(s) 119). One or more sources may include one or more of: publicly available indicators; MBSF; LDS SAF; Medicare outpatient limited data set standard analytical files; Medicare Skilled Nursing Facility (SNF) limited data set standard analytical files; HCAHPS; Orthopedic Board Certification Data; or total volume data from the American Hospital Directory (AHD). By integrating these diverse data sources, healthcare organizations may gain a comprehensive understanding of performance across multiple dimensions, such as clinical outcomes, patient satisfaction, resource utilization, and adherence to best practices, ultimately supporting more informed decision-making and quality improvement initiatives.

In step 203, the analysis platform 101 may select, based on the plurality of data, one or more procedures and/or conditions upon which performance evaluation of one or more entities (e.g., hospital) is based. In one instance, one or more procedures and/or conditions may be selected based on one or more of: a frequency of admission, an ability to make entity-to-entity comparisons, or a presence of a sufficient degree of risk or complexity such that a quality of an entity's performance is important. In one example, procedures and/or conditions that may have a high frequency of admission may be prioritized. These may be common medical events that may significantly impact a larger number of patients. By focusing on frequently occurring procedures and conditions, the evaluation may address areas that are critical to the majority of patients and may provide insights into the overall quality and care delivered by hospitals. In one example, procedures and/or conditions may be chosen based on their suitability for making meaningful comparisons between different entities (e.g., hospitals). This may mean selecting conditions that may be treated similarly across various hospitals, allowing for standardized comparisons. These procedures and/or conditions may have well-defined diagnostic and treatment protocols that may minimize variability and enable accurate performance benchmarking across hospitals. In one example, the selected procedures and/or conditions may entail a sufficient degree of risk or complexity to make the evaluation of performance meaningful. Procedures and/or conditions with higher risk or complexity may require more advanced skill, specialized equipment, and coordinated care. Evaluating performance in these areas highlighting the capability of hospitals to manage challenging and resource-intensive cases, may provide a clearer picture of their quality and effectiveness.

In one instance, the one or more procedures and/or conditions are selected based on an inclusion criteria and/or an exclusion criteria. The inclusion criteria and/or the exclusion criteria are defined based on one or more of maximal homogeneity, maximal sample size, or minimal coding variation. In one example, the inclusion and exclusion criteria may aim to select procedures and/or conditions that may exhibit uniformity across different hospitals. Homogeneity may reduce variability due to differences in patient populations, treatment protocols, or hospital practices. In one example, procedures and/or conditions may be chosen to maximize the sample size, ensuring that a sufficient number of cases are available for robust statistical analysis. Larger sample sizes may enhance the reliability of the finding, may reduce the margin of error, and may increase the generalizability of the result. In one example, criteria may aim to minimize coding variation, which may refer to discrepancies in how medical procedures and/or conditions may be documented across different entities. Consistent and accurate coding may be essential for reliable data analysis and comparison. In one instance, the one or more entities may be selected by excluding, from a plurality of entities associated with a database, each entity that is associated with one or more attributes indicative of non-inclusion. To ensure that the evaluation process accurately reflects the performance of entities (e.g., hospitals), it may be necessary to exclude certain entities from the assessment based on specific attributes indicative of non-inclusion. This step may include a filtering process where entities that do not meet predefined criteria are excluded from the pool of entities under consideration.

The one or more attributes may include one or more of: an absence of Medicare provider number; a clinically-integrated facility; a primary service (SERV) code indicating a service type other than a specific set of conditions; or a volume insufficient to allow estimation for at least one outcome. In one example, entities lacking a Medicare provider number may be excluded from evaluation. Medicare provider number is a unique identifier for hospitals participating in the Medicare program. Its absence may indicate that the entity is not involved in Medicare services, thereby limiting the comparability of data and outcomes with other entities that are Medicare providers. In one example, facilities that are not clinically integrated may be excluded. Clinical integration may refer to the coordination of patient care across different services and providers within a hospital. Non-integrated facilities may have fragmented care processes, making it difficult to assess performance consistently. In one example, hospitals with a primary service code indicating a service type that does not fall within the specified set of conditions for evaluation may be excluded. The primary service code may classify the main type of care provided by a hospital, such as general medical/surgical, pediatric, or psychiatric. If the evaluation focuses on general medical/surgical care, hospitals providing psychiatric services may be excluded. In one example, entities with insufficient patient volume to allow reliable estimation of outcomes may be excluded. Adequate patient volume is essential for statistical analysis, as it ensures the robustness and reliability of the performance metrics.

In step 205, the analysis platform 101 may select, based on the plurality of data, one or more entities for which the performance evaluation is conducted. In one example, the selection may aim to identify hospitals that meet predefined criteria for inclusion in the evaluation. By analyzing a diverse range of data sources, the selection process ensures that chosen entities are representative and suitable for rigorous performance assessment.

In step 207, the analysis platform 101 may, for each of one or more procedures and/or conditions, determine, using one or more models, a performance score for each of the one or more selected entities based on one or more performance indicators. The one or more performance indicators may include risk-adjusted outcome(s), process measure(s), and structural measure(s). In one instance, the one or more models may be selected by evaluating model statistics for combinations of performance indicators. This may involve assessing various statistical metrics and criteria to determine the model's suitability for accurately measuring healthcare performances. Model(s) may be scrutinized based on their ability to integrate and weight performance indicators effectively, ensuring robustness in evaluating outcomes, process adherence, and structural capabilities of healthcare entities. By evaluating model statistics, such as goodness of fit, predictive accuracy, and sensitivity to different data distributions, the chosen model may be optimized to provide comprehensive and reliable insights into healthcare quality.

The analysis platform 101 may determine the one or more models associated with an optimal combination of one or more of: a number of performance indicators, a model fit, or consistency with models in related cohorts. This may involve selecting models that demonstrate robust statistical performance in integrating multiple performance indicators effectively. In one example, model fit may refer to how well a statistical model aligns with observed data, ensuring that it accurately captures the relationship between variables. In one example, consistency across related cohorts may ensure that selected models maintain reliability and applicability across different patient populations or healthcare settings. By prioritizing models with optimal combinations of performance indicators and ensuring they exhibit strong fit and consistency, healthcare evaluations can reliably assess outcomes.

In one instance, the one or more risk-adjusted outcomes have been risk-adjusted using a multi-level logistic regression model. In one instance, the multi-level logistic regression model may operate by analyzing data hierarchically, considering both individual-level characteristics (such as patient demographics and clinical conditions) and high-level factors (such as hospital characteristics or regional variations). By employing this approach the model adjusts outcome measure to reflect differences in patient risk profiles, ensuring fair comparisons across healthcare entities.

In one instance, the one or more risk-adjusted outcomes have been risk-adjusted based on one or more risk-adjustment variables. The one or more risk-adjustment variables may include one or more: inbound transfer status; year of hospital admission; Elixhauser comorbidities; Medicare status code; socioeconomic status; condition cohort-specific covariates; surgical cohort-specific covariates; history of stroke; or Covid-19 diagnosis. By incorporating these variables into the risk-adjustment process, statistical model(s) may normalize outcome measures across different patient populations and healthcare settings. This adjustment may ensure that comparisons between healthcare entities are fair and meaningful, as it may account for the diverse patient profiles and underlying health risks encountered in clinical practice. Adjusting outcomes based on these variables may enhance the accuracy and reliability of performance assessments.

In one instance, the one or more risk-adjusted outcomes may include one or more of: mortality within a pre-determined time period; unplanned readmission within a pre-determined time period; surgical site infection, hip replacement, knee replacement, Abdominal Aortic Aneurysm Repair (AAA), Heart Bypass Surgery (CABG), and Aortic Valve Surgery (AVR) cohorts; revision within a pre-determined time period, hip replacement, and knee replacement cohorts; prolonged hospitalization, leukemia, lymphoma and myeloma and procedure cohorts; discharge to a location other than the patient's home; stroke on procedure date, CABG, AVR, and Transcatheter Aortic Valve Replacement (TAVR) cohorts; or time spent at home within a pre-determined time period of discharge.

In one instance, the one or more process measures may include one or more of: worker flu immunization; noninvasive ventilation; patient experience; board certification; emergency room visits after chemotherapy; unplanned visits after colonoscopy; compliance with the septic shock bundle; or public transparency. There process measures may help evaluate different facets of healthcare quality, emphasizing aspects like patient safety, clinical effectiveness, patient-centered care, and provider competence.

In one instance, the one or more structural measures may include one or more of: volume of procedures; nurse staffing; or National Cancer Institute (NCI)-designated Cancer Center and/or American College of Surgeons (ACS) Commission on Cancer. As discussed, structural measures in healthcare evaluation may encompass critical indicators that assess the resources, capabilities, and organizational features of healthcare facilities. In one example, evaluating the ratio of registered nurses to patient volume or adjusted patient days, which may impact patient safety. Adequate nurse staffing levels are associated with improved patient care. Structural measures may provide insights into the capacity and readiness of hospitals to deliver high-quality care across various specialties and conditions. By integrating structural measures with other performance indicators like process and outcome measures, a comprehensive view of healthcare quality is provided.

In step 209, the analysis platform 101 may the analysis platform 101 may generate a rank for each of the one or more selected entities based on the performance score determined for each of the one or more selected entities. The performance scores may be derived from comprehensive assessments of healthcare entities across various performance indicators, including risk-adjusted outcomes, process measures, and structural measures. The ranking process may aggregate these scores to create a hierarchical order, where entities demonstrating superior performance across the evaluated criteria may achieve higher ranks. This ranking system may provide a clear and objective representation of healthcare quality, enabling comparison among entities.

In step 211, the analysis platform 101 may cause a display of the rank for one or more selected entities in association with one or more procedures and/or conditions in a user interface of a device. In one instance, the user interface may integrate performance scores and ranks derived from evaluations across various healthcare quality indicators, ensuring transparency and usability. The users may interact with the interface to view detailed rankings and, compare performance across different entities. By visualizing the data effectively, the user interface facilitates informed decision-making.

FIG. 2B is a flowchart of a process for dynamically ranking one or more entities by leveraging comprehensive quality assessment, according to aspects of the disclosure. In various embodiments, the analysis platform 101 and/or any of the modules 103-113 may perform one or more portions of the process 212 and are implemented using, for instance, a chip set including a processor and a memory as shown in FIG. 7. As such, the analysis platform 101 and/or any of modules 103-113 may provide means for accomplishing various parts of the process 212, as well as means for accomplishing embodiments of other processes described herein in conjunction with other components of the system 100. Although the process 212 is illustrated and described as a sequence of steps, it is contemplated that various embodiments of the process 212 may be performed in any order or combination and need not include all of the illustrated steps.

In step 213, the analysis platform 101 may collect a plurality of data from one or more sources. In one example, the analysis platform 101 may retrieve publicly available indicators, such as national healthcare statistics, and specialized datasets like MBSF and LDS SAF. Additionally, the analysis platform 101 may encompass clinical outcomes data, patient surveys (e.g., HCAGPS), administrative records (e.g., AHA annual surveys), and specialty-specific board certification information. The integration of these varied data points may ensure a holistic view of hospital performance, allowing for more accurate and reliable quality assessment by capturing a wide range of metrics and perspectives relevant to healthcare delivery.

In step 215, the analysis platform 101 may process the plurality of data for evaluating the performance of one or more entities (e.g., hospitals) across a plurality of procedures and/or conditions. In one instance, the analysis platform 101, via the data processing module 103, may perform data cleaning to ensure accuracy, normalization to standardize different data formats, and risk adjustment to account for patient demographics and health conditions. For example, the performance data may be normalized across demographics and health conditions based on one or more variables (e.g., age, gender, comorbidities, or socioeconomic status). In one example, advanced statistical and machine-learning algorithms may be applied to dynamically weight quality indicators, identifying patterns and correlations that influence outcomes. The data is then categorized and evaluated across multiple procedures and/or conditions to assess performance comprehensively, such as AAA, AVR, COPD, Colon cancer surgery, CHF, Diabetes, Back surgery (Spinal fusion), Heart attack, CABG, Hip fracture, Hip replacement, Kidney failure, Knee replacement, Leukemia, lymphoma & myeloma, Lung cancer surgery, Ovarian cancer surgery, Pneumonia, Prostate cancer surgery, Stroke, TAVR, and Uterine cancer surgery. By integrating and analyzing this extensive data, the analysis platform 101 may provide nuanced insights into hospital performance.

In one instance, the analysis platform 101 may dynamically adjust, utilizing a machine-learning model, a weighting of quality indicators during the ranking of the entities. The weighting is dynamically adjusted based on measurement errors or incomplete risk adjustment during the ranking. In one instance, quality indicators may encompass measurable aspects of healthcare processes, outcomes, patient perceptions, and organizational structures that gauge the effectiveness, safety, patient-centeredness, efficiency, and equity of healthcare delivery. For example, process indicators may assess adherence to clinical guidelines and protocols, ensuring consistent and standardized care practices. For example, outcome indicators may measure the result of healthcare interventions on patient health and functional status, including mortality rates, complication rates, and patient-reported outcomes. For example, structure indicators may evaluate the resources and infrastructure supporting healthcare delivery, such as staffing levels and facility accreditation. For example, patient experience indicators may capture patient satisfaction and perceptions of care quality.

In one instance, the machine-learning model may continuously update the weighting factors based on real-time data from one or more sources. In one instance, the analysis platform 101 may assign a higher weight to at least one quality indicator associated with a critical health outcome, wherein the critical health outcome includes patient safety or treatment effectiveness

In step 217, the analysis platform 101 may determine point assignment criteria or point deduction criteria for entities (e.g., hospitals) based on their performance across the plurality of procedures and/or conditions. In this disclosure, one or both of the point assignment criteria or point deduction criteria may be referred to as “point criteria.” In one instance, the analysis platform 101 may develop specific metrics and thresholds for performance evaluation in each specialty and procedure, such as mortality rates, complication rates, patient satisfaction scores, adherence to clinical guidelines, and success rates of specific surgeries or treatments. The analysis platform 101 may award points to hospitals that excel in these metrics demonstrating high performance in specialties like cardiology, oncology, orthopedics, neurology, and general surgery, and procedures such as CABG, knee and hip replacements, cancer surgeries, stroke treatments, and diabetes management. In one example, the analysis platform 101 may determine the performance of the hospitals across the plurality of procedures and/or conditions is above a pre-determined threshold level, and may assign points to the hospitals for each specialty and procedure where the performance is above the pre-determined threshold level. Conversely, the analysis platform 101 may deduct points for underperformance or failure to meet established threshold levels. In one example, the analysis platform 101 may determine the performance of the hospitals across the plurality of procedures and/or conditions is below a pre-determined threshold level, and may deduct a point from the hospitals for each specialty and procedure where the performance is below the pre-determined threshold level. The criteria may be empirically derived using advanced statistical and machine-learning techniques to ensure accuracy and fairness, accounting for factors like patient demographics and case complexity. This rigorous point-based system may enable a transparent and equitable ranking of hospitals.

In step 219, the analysis platform 101 may generate a rank for each of the entities (e.g., hospitals) based on aggregated points derived from the performance data of one or more entities. As previously discussed, each hospital's performance may be evaluated against pre-defined criteria, and points may be assigned or deducted based on these evaluations, reflecting the hospital's proficiency in delivering quality healthcare across diverse medical fields. The analysis platform 101 may employ advanced algorithms and statistical models to aggregate the points into a composite score. The analysis platform 101 may also utilize weighted averages, where different procedures and/or conditions may contribute differently based on their significance and impact on overall healthcare outcomes. The analysis platform 101 may also utilize machine-learning algorithms to dynamically adjust weights based on empirical data analysis, ensuring that the ranking reflects current and relevant performance trends. Once aggregated, hospitals are ranked ordinally based on their composite score, providing a precise hierarchical ordering. In one example, analysis platform 101 may assign a state-specific rank to an entity upon comparing the ranking of the entity to one or more entities within the same state. In one example, analysis platform 101 may assign a metro-specific rank to an entity upon comparing the ranking of the entity to one or more entities within the same metropolitan area.

In step 221, the analysis platform 101 may cause a display of the ranking (e.g., the rank for each of the one or more entities) in the user interface of a device. In one instance, the analysis platform 101 may design an intuitive user interface that presents the hospital rankings based on composite scores derived from comprehensive performance metrics across procedures and/or conditions. The interface may employ interactive charts, graphs, and tables to allow the users to easily navigate and interpret the data. Advanced visualization techniques may ensure clarity and accessibility, enabling users to compare hospital rankings, identify trends, and make informed decisions regarding healthcare quality and provider selection. Additionally, the interface may support customization options, filters, and real-time updates to accommodate varying user needs and maintain relevance in the healthcare decision-making process.

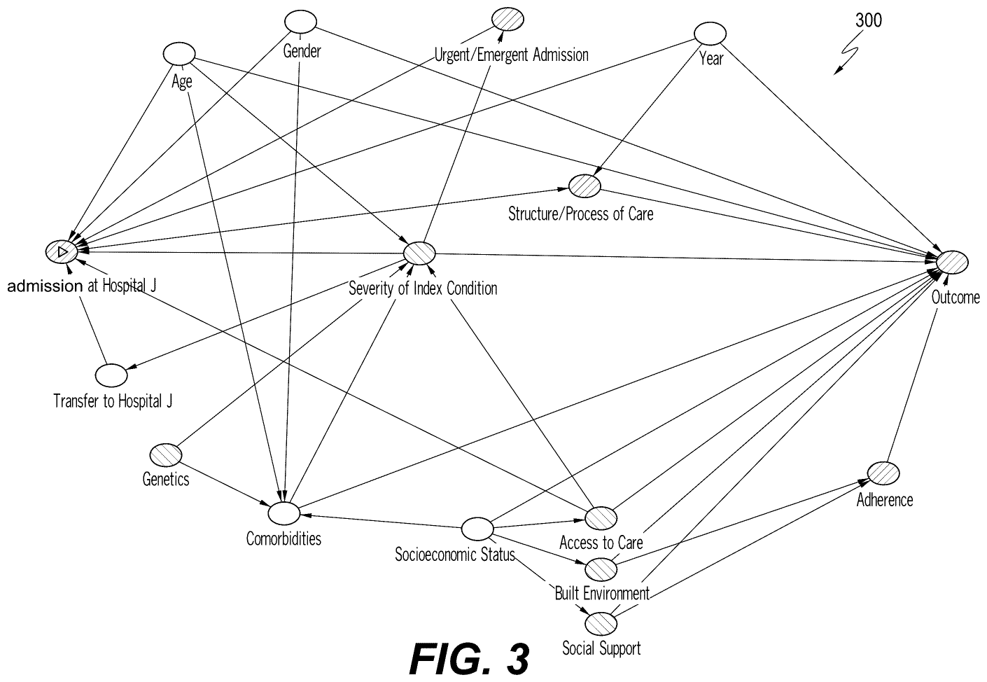

FIG. 3 illustrates an acyclic graph that shows the hypothesized relationship between covariates, hospital selection, and outcomes, according to aspects of the disclosure. In one instance, the analysis platform 101, via visualization module 109, may generate an acyclic graph 300 in the user interface of a device. In this example, the acyclic graph 300 includes:
1. Admission at Hospital J: Admission at a specific hospital (e.g., hospital J) may be influenced by the severity of the patient's condition and whether the admission is urgent or an emergency. It may also be connected to patients being transferred from another facility to hospital J.
2. Transfer to Hospital J: Transfer to Hospital J may be determined by the severity of the patient's condition, indicating that specialized care or a higher level of treatments may be required at Hospital J.
3. Age: Age may influence the presence of comorbidities and the severity of the primary condition, which in turn may affect the outcome of the treatment.
4. Genetics: Genetic factors may play a role in the development of comorbidities and the severity of diseases, impacting overall health conditions.
5. Gender: Gender may influence the prevalence of certain comorbidities and the severity of specific conditions, which may subsequently affect outcomes.
6. Comorbidities: The presence of additional diseases or conditions may worsen the severity of the primary condition and negatively impact health outcomes.
7. Urgent or emergency admission: An urgent or emergency admission may indicate a higher severity of the primary condition, influencing the likelihood of admission at Hospital J and affecting outcomes.
8. Severity of index condition: The severity of the primary condition may be influenced by age, genetics, gender, and comorbidities. It may affect whether a patient is admitted to or transferred to Hospital J and directly impacts health outcomes.
9. Year: The year may reflect advancements in medical technology, treatments, and care processes, which may influence the structure and process of care at hospitals and ultimately impact patient outcomes.
10. Structure/Process of care: The organization and execution of healthcare services may be influenced by the year, reflecting changes and improvements over time. This node may directly affect health outcomes.
11. Socioeconomic status: Socioeconomic status may affect access to healthcare services, the quality of the built environment, social support networks, and patient adherence to treatment plans, all of which may influence health outcomes.
12. Access to care: The ability to obtain healthcare services may be influenced by socioeconomic status and may have a direct impact on health outcomes.
13. Built environment: The physical and infrastructural aspects of a patient's environment may be influenced by socioeconomic status and may affect health outcomes.
14. Social support: The level of support available to a patient may be influenced by socioeconomic status and may impact health outcomes by providing emotional and practical assistance.
15. Adherence: Patient's adherence to treatment plans and medical advice may be influenced by socioeconomic status and may directly affect health outcomes.
16. Outcome: The final health outcome may be the result of a complex interplay of multiple factors, including patient demographics (e.g., age, gender, genetics), health status (e.g., comorbidities, severity of condition), healthcare system factors (year, structure/process of care), and social determinants (e.g., socioeconomic status, access to care, built environment, social support, adherence).

In one instance, this detailed breakdown may highlight the hypothesized relationships among various covariates, hospital selection criteria, and health outcomes, emphasizing the complexity and interconnectedness of factors influencing patient care and hospital performance.

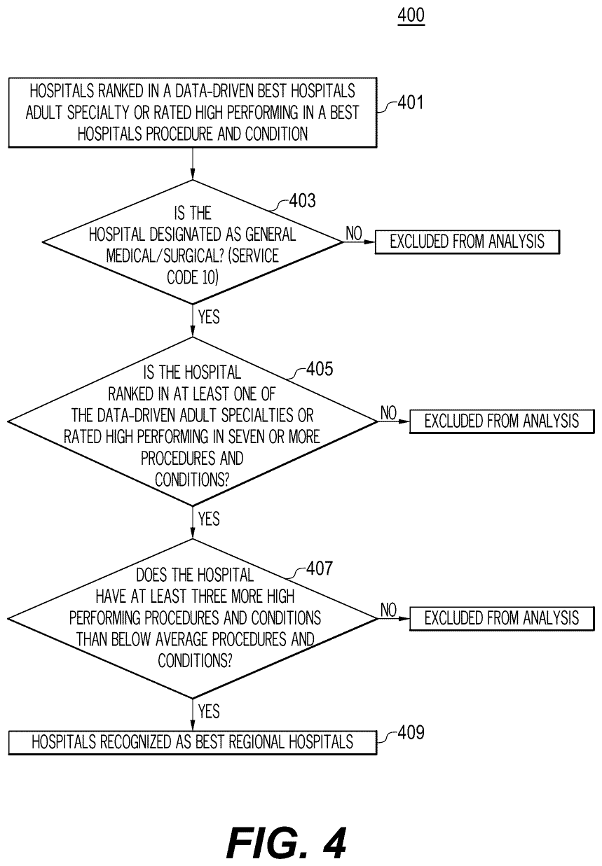

FIG. 4 is a flowchart of a process for determining whether an entity (e.g., hospital) is recognized as the best service provider, according to aspects of the disclosure. In various embodiments, the analysis platform 101 and/or any of the modules 103-113 may perform one or more portions of the process 400 and are implemented using, for instance, a chip set including a processor and a memory as shown in FIG. 7. As such, the analysis platform 101 and/or any of modules 103-113 may provide means for accomplishing various parts of the process 400, as well as means for accomplishing embodiments of other processes described herein in conjunction with other components of the system 100. Although the process 400 is illustrated and described as a sequence of steps, it is contemplated that various embodiments of the process 400 may be performed in any order or combination and need not include all of the illustrated steps.

In step 401, the analysis platform 101 may identify hospitals that are ranked in at least one adult specialty (e.g., cardiology or oncology) based on data-driven metrics. Additionally, hospitals that have been rated as high-performing in specific procedures and conditions (e.g., knee replacement) are also considered. This step may ensure that only hospitals with proven excellence in specialized care or specific high-performing procedures are included in the initial pool for further analysis.

In step 403, the analysis platform 101 may check whether one or more hospitals are classified as a general medical/surgical hospital, as indicated by Service Code 10. If a hospital does not fall under the general medical/surgical category, then the hospital is excluded from further analysis. Examples of excluded hospitals may include specialty hospitals, rehabilitation centers, or psychiatric hospitals. If the hospital is a general medical/surgical hospital, then proceed to the next step for further evaluation.

In step 405, the analysis platform 101 may evaluate the hospital's performance breadth and depth. The hospital must be either ranked in at least one adult specialty or have a high performance in multiple (e.g., at least seven) specific procedures and conditions. If the hospital does not meet the required threshold for specialty or procedural performance, the hospital is excluded from further analysis. If the hospital demonstrates significant performance in specialized care or multiple procedures and conditions, proceed to the next step for further evaluation.

In step 407, the analysis platform 101 may assess the hospital's overall balance of performance. The hospital must have a net positive performance, meaning it should have at least three more high-performing areas compared to areas rated below average. If the hospital's performance does not sufficiently outweigh its below-average ratings, the hospital is excluded from further analysis. If the hospital has a net positive performance balance, the hospital qualifies for the final designation.

In step 409, the analysis platform 101 may designate a hospital as the best upon passing all the previous criteria. This recognition may indicate that the hospital is among the top-performing hospitals in its region, based on comprehensive and rigorous evaluation criteria. Acknowledging hospitals that excel in providing high-quality medical and surgical care across multiple procedures and/or conditions, ensures they stand out in their respective region for their exceptional performance.

By following these steps, the analysis platform 101 may ensure that only the most qualified hospitals, those demonstrating consistently high performance across various procedures and/or conditions, are recognized as the best. This rigorous evaluation may facilitate maintaining high standards and provide valuable insights to patients and healthcare stakeholders.

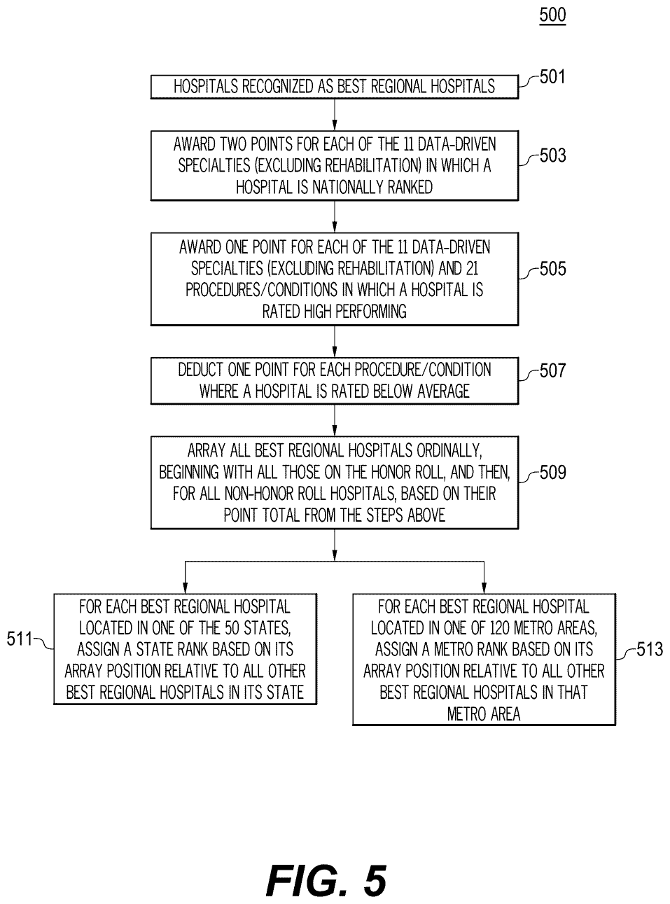

FIG. 5 is a flowchart of a process for a scoring methodology that determines the state and metro area rankings of entities (e.g., hospitals), according to aspects of the disclosure. In various embodiments, the analysis platform 101 and/or any of the modules 103-113 may perform one or more portions of the process 500 and are implemented using, for instance, a chip set including a processor and a memory as shown in FIG. 7. As such, the analysis platform 101 and/or any of modules 103-113 may provide means for accomplishing various parts of the process 500, as well as means for accomplishing embodiments of other processes described herein in conjunction with other components of the system 100. Although the process 500 is illustrated and described as a sequence of steps, it is contemplated that various embodiments of the process 500 may be performed in any order or combination and need not include all of the illustrated steps.

In step 501, the analysis platform 101 may identify hospitals that are considered top-performing within their respective regions. These hospitals may be selected based on pre-defined criteria that may include overall quality, patient outcomes, and reputation.

In step 503, the analysis platform 101 may assign two points to the hospitals for each specialty (from a list of 11) in which they are nationally ranked. In one example, if a hospital is recognized at the national level in specialties like cardiology, neurology, or orthopedics, they are assigned two points per specialty.

In step 505, the analysis platform 101 may assign one point to the hospitals for each specialty and each of the 21 specific procedures or conditions where they are rated as high-performing. This recognizes the hospitals that excel in particular areas of treatment and patient care.

In step 507, the analysis platform 101 may deduct points for each procedure or condition where the hospital's performance is rated below average. The analysis platform 101 may penalize hospitals for poor performance in specific areas, ensuring that only consistently high-performing hospitals rank higher.

In step 509, the analysis platform 101 may order hospitals based on whether they are on the honor roll, a list of the top hospitals based on comprehensive performance metrics. After honor roll hospitals, the remaining hospitals are arrayed based on their total points from the previous steps, creating an ordinal ranking.

In step 511, the analysis platform 101 may rank hospitals within their respective stats based on their position in the overall array. The analysis platform 101 may assign a state-specific rank to each hospital, allowing for comparison within the same state.

In step 513, the analysis platform 101 may assign a metro-specific rank to the hospitals based on their position relative to other hospitals in the same metropolitan area. This step may ensure that hospitals are compared and ranked within their local metro contexts.

By following these steps, the flow chart outlines a comprehensive and methodical approach to ranking hospitals based on a combination of national recognition, specialty performance, specific procedures, and geographical positioning, ensuring a fair and detailed assessment of hospital quality.

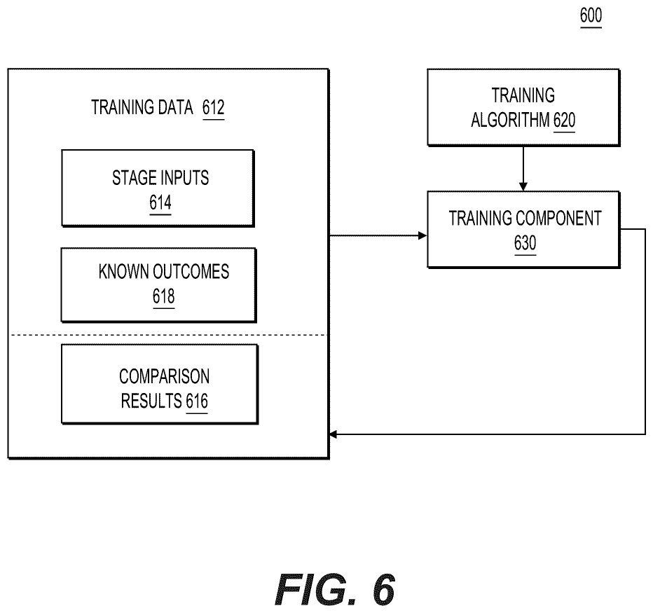

One or more implementations disclosed herein include and/or may be implemented using a machine-learning model. For example, one or more of the modules of the analysis platform 101 may be implemented using a machine-learning model and/or may be used to train the machine-learning model. A given machine-learning model may be trained using the training flow chart 600 of FIG. 6. Training data 612 may include one or more of stage inputs 614 and known outcomes 618 related to the machine-learning model to be trained. The stage inputs 614 may be from any applicable source including text, visual representations, data, values, comparisons, stage outputs, e.g., one or more outputs from one or more actions or operations from FIGS. 2A-2B. The known outcomes 618 may be included for the machine-learning models generated based upon supervised or semi-supervised training. An unsupervised machine-learning model may not be trained using known outcomes 618. Known outcomes 618 may include known or desired outputs for future inputs similar to, or in the same category as, stage inputs 614 that do not have corresponding known outputs.

The training data 612 and a training algorithm 620, e.g., one or more of the modules implemented using the machine-learning model and/or may be used to train the machine-learning model, may be provided to a training component 630 that may apply the training data 612 to the training algorithm 620 to generate the machine-learning model. According to an implementation, the training component 630 may be provided comparison results 616 that compare a previous output of the corresponding machine-learning model to apply the previous result to re-train the machine-learning model. The comparison results 616 may be used by training component 630 to update the corresponding machine-learning model. The training algorithm 620 may utilize machine-learning networks and/or models including, but not limited to a deep learning network such as Deep Neural Networks (DNN), Convolutional Neural Networks (CNN), Fully Convolutional Networks (FCN) and Recurrent Neural Networks (RCN), probabilistic models such as Bayesian Networks and Graphical Models, classifiers such as K-Nearest Neighbors, and/or discriminative models such as Decision Forests and maximum margin methods, models specifically discussed in the present disclosure, or the like.

The machine-learning model used herein may be trained and/or used by adjusting one or more weights and/or one or more layers of the machine-learning model. For example, during training, a given weight may be adjusted (e.g., increased, decreased, removed) based upon training data or input data. Similarly, a layer may be updated, added, or removed based upon training data/and or input data. The resulting outputs may be adjusted based upon the adjusted weights and/or layers.

In general, any process or operation discussed in this disclosure is understood to be computer-implementable, such as the processes illustrated in FIG. 2A-2B may be performed by one or more processors of a computer system as described herein. A process or process step performed by one or more processors may also be referred to as an operation. The one or more processors may be configured to perform such processes by having access to instructions (e.g., software or computer-readable code) that, when executed by one or more processors, cause the one or more processors to perform the processes. The instructions may be stored in a memory of the computer system. A processor may be a central processing unit (CPU), a graphics processing unit (GPU), or any suitable type of processing unit.

A computer system, such as a system or device implementing a process or operation in the examples above, may include one or more computing devices. One or more processors of a computer system may be included in a single computing device or distributed among a plurality of computing devices. One or more processors of a computer system may be connected to a data storage device. A memory of the computer system may include the respective memory of each computing device of the plurality of computing devices.

FIG. 7 illustrates an implementation of a computer system that may execute techniques presented herein. The computer system 700 can include a set of instructions that can be executed to cause the computer system 700 to perform any one or more of the methods or computer based functions disclosed herein. The computer system 700 may operate as a standalone device or may be connected, e.g., using a network, to other computer systems or peripheral devices.

Unless specifically stated otherwise, as apparent from the following discussions, it is appreciated that throughout the specification, discussions utilizing terms such as “processing,” “computing,” “calculating,” “determining”, analyzing” or the like, refer to the action and/or processes of a computer or computing system, or similar electronic computing device, that manipulate and/or transform data represented as physical, such as electronic, quantities into other data similarly represented as physical quantities.

In a similar manner, the term “processor” may refer to any device or portion of a device that processes electronic data, e.g., from registers and/or memory to transform that electronic data into other electronic data that, e.g., may be stored in registers and/or memory. A “computer,” a “computing machine,” a “computing platform,” a “computing device,” or a “server” may include one or more processors.

In a networked deployment, the computer system 700 may operate in the capacity of a server or as a client user computer in a server-client user network environment, or as a peer computer system in a peer-to-peer (or distributed) network environment. The computer system 700 can also be implemented as or incorporated into various devices, such as a personal computer (PC), a tablet PC, a set-top box (STB), a personal digital assistant (PDA), a mobile device, a palmtop computer, a laptop computer, a desktop computer, a communications device, a wireless telephone, a land-line telephone, a control system, a camera, a scanner, a facsimile machine, a printer, a pager, a personal trusted device, a web appliance, a network router, switch or bridge, or any other machine capable of executing a set of instructions (sequential or otherwise) that specify actions to be taken by that machine. In a particular implementation, the computer system 700 can be implemented using electronic devices that provide voice, video, or data communication. Further, while the computer system 700 is illustrated as a single system, the term “system” shall also be taken to include any collection of systems or sub-systems that individually or jointly execute a set, or multiple sets, of instructions to perform one or more computer functions.

As illustrated in FIG. 7, the computer system 700 may include a processor 702, e.g., a central processing unit (CPU), a graphics processing unit (GPU), or both. The processor 702 may be a component in a variety of systems. For example, the processor 702 may be part of a standard personal computer or a workstation. The processor 702 may be one or more processors, digital signal processors, application specific integrated circuits, field programmable gate arrays, servers, networks, digital circuits, analog circuits, combinations thereof, or other now known or later developed devices for analyzing and processing data. The processor 702 may implement a software program, such as code generated manually (i.e., programmed).

The computer system 700 may include a memory 704 that can communicate via bus 708. The memory 704 may be a main memory, a static memory, or a dynamic memory. The memory 704 may include, but is not limited to computer readable storage media such as various types of volatile and non-volatile storage media, including but not limited to random access memory, read-only memory, programmable read-only memory, electrically programmable read-only memory, electrically erasable read-only memory, flash memory, magnetic tape or disk, optical media and the like. In one implementation, the memory 704 includes a cache or random-access memory for the processor 702. In alternative implementations, the memory 704 is separate from the processor 702, such as a cache memory of a processor, the system memory, or other memory. The memory 704 may be an external storage device or database for storing data. Examples include a hard drive, compact disc (“CD”), digital video disc (“DVD”), memory card, memory stick, floppy disc, universal serial bus (“USB”) memory device, or any other device operative to store data. The memory 704 is operable to store instructions executable by the processor 702. The functions, acts or tasks illustrated in the figures or described herein may be performed by the processor 702 executing the instructions stored in the memory 704. The functions, acts, or tasks are independent of the particular type of instruction set, storage media, processor or processing strategy and may be performed by software, hardware, integrated circuits, firmware, micro-code and the like, operating alone or in combination. Likewise, processing strategies may include multiprocessing, multitasking, parallel processing, and the like.

As shown, the computer system 700 may further include a display 710, such as a liquid crystal display (LCD), an organic light emitting diode (OLED), a flat panel display, a solid-state display, a cathode ray tube (CRT), a projector, a printer or other now known or later developed display device for outputting determined information. The display 710 may act as an interface for the user to see the functioning of the processor 702, or specifically as an interface with the software stored in the memory 704 or in the drive unit 706.

Additionally or alternatively, the computer system 700 may include an input/output device 712 configured to allow a user to interact with any of the components of the computer system 700. The input/output device 712 may be a number pad, a keyboard, or a cursor control device, such as a mouse, or a joystick, touch screen display, remote control, or any other device operative to interact with the computer system 700.

The computer system 700 may also or alternatively include drive unit 706 implemented as a disk or optical drive. The drive unit 706 may include a computer-readable medium 722 in which one or more sets of instructions 724, e.g. software, can be embedded. Further, instructions 724 may embody one or more of the methods or logic as described herein. The instructions 724 may reside completely or partially within the memory 704 and/or within the processor 702 during execution by the computer system 700. The memory 704 and the processor 702 also may include computer-readable media as discussed above.

In some systems, computer-readable medium 722 includes the set of instructions 724 or receives and executes the set of instructions 724 responsive to a propagated signal so that a device connected to network 730 can communicate voice, video, audio, images, or any other data over the network 730. Further, the set of instructions 724 may be transmitted or received over the network 730 via communication port or interface 720, and/or using bus 708. The communication port or interface 720 may be a part of the processor 702 or may be a separate component. The communication port or interface 720 may be created in software or may be a physical connection in hardware. The communication port or interface 720 may be configured to connect with a network 730, external media, the display 710, or any other components in computer system 700, or combinations thereof. The connection with the network 730 may be a physical connection, such as a wired Ethernet connection or may be established wirelessly as discussed below. Likewise, the additional connections with other components of the computer system 700 may be physical connections or may be established wirelessly. The network 730 may alternatively be directly connected to the bus 708.

While the computer-readable medium 722 is shown to be a single medium, the term “computer-readable medium” may include a single medium or multiple media, such as a centralized or distributed database, and/or associated caches and servers that store one or more sets of instructions. The term “computer-readable medium” may also include any medium that is capable of storing, encoding, or carrying a set of instructions for execution by a processor or that causes a computer system to perform any one or more of the methods or operations disclosed herein. The computer-readable medium 722 may be non-transitory, and may be tangible.

The computer-readable medium 722 can include a solid-state memory such as a memory card or other package that houses one or more non-volatile read-only memories. The computer-readable medium 722 can be a random-access memory or other volatile re-writable memory. Additionally or alternatively, the computer-readable medium 722 can include a magneto-optical or optical medium, such as a disk or tapes or other storage device to capture carrier wave signals such as a signal communicated over a transmission medium. A digital file attachment to an e-mail or other self-contained information archive or set of archives may be considered a distribution medium that is a tangible storage medium. Accordingly, the disclosure is considered to include any one or more of a computer-readable medium or a distribution medium and other equivalents and successor media, in which data or instructions may be stored.

In an alternative implementation, dedicated hardware implementations, such as application specific integrated circuits, programmable logic arrays and other hardware devices, can be constructed to implement one or more of the methods described herein. Applications that may include the apparatus and systems of various implementations can broadly include a variety of electronic and computer systems. One or more implementations described herein may implement functions using two or more specific interconnected hardware modules or devices with related control and data signals that can be communicated between and through the modules, or as portions of an application-specific integrated circuit. Accordingly, the present system encompasses software, firmware, and hardware implementations.

Computer system 700 may be connected to network 730. The network 730 may define one or more networks including wired or wireless networks. The wireless network may be a cellular telephone network, an 802.10, 802.16, 802.20, or WiMAX network. Further, such networks may include a public network, such as the Internet, a private network, such as an intranet, or combinations thereof, and may utilize a variety of networking protocols now available or later developed including, but not limited to TCP/IP based networking protocols. The network 730 may include wide area networks (WAN), such as the Internet, local area networks (LAN), campus area networks, metropolitan area networks, a direct connection such as through a Universal Serial Bus (USB) port, or any other networks that may allow for data communication. The network 730 may be configured to couple one computing device to another computing device to enable communication of data between the devices. The network 730 may generally be enabled to employ any form of machine-readable media for communicating information from one device to another. The network 730 may include communication methods by which information may travel between computing devices. The network 730 may be divided into sub-networks. The sub-networks may allow access to all of the other components connected thereto or the sub-networks may restrict access between the components. The network 730 may be regarded as a public or private network connection and may include, for example, a virtual private network or an encryption or other security mechanism employed over the public Internet, or the like.

In accordance with various implementations of the present disclosure, the methods described herein may be implemented by software programs executable by a computer system. Further, in an example, non-limited implementation, implementations can include distributed processing, component/object distributed processing, and parallel processing. Alternatively, virtual computer system processing can be constructed to implement one or more of the methods or functionality as described herein.

Although the present specification describes components and functions that may be implemented in particular implementations with reference to particular standards and protocols, the disclosure is not limited to such standards and protocols. For example, standards for Internet and other packet switched network transmission (e.g., TCP/IP, UDP/IP, HTML, HTTP) represent examples of the state of the art. Such standards are periodically superseded by faster or more efficient equivalents having essentially the same functions. Accordingly, replacement standards and protocols having the same or similar functions as those disclosed herein are considered equivalents thereof.

It will be understood that the steps of methods discussed are performed in one embodiment by an appropriate processor (or processors) of a processing (i.e., computer) system executing instructions (computer-readable code) stored in storage. It will also be understood that the disclosure is not limited to any particular implementation or programming technique and that the disclosure may be implemented using any appropriate techniques for implementing the functionality described herein. The disclosure is not limited to any particular programming language or operating system.

It should be appreciated that in the above description of example embodiments of the invention, various features of the invention are sometimes grouped together in a single embodiment, figure, or description thereof for the purpose of streamlining the disclosure and aiding in the understanding of one or more of the various inventive aspects. This method of the present disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

In the description provided herein, numerous specific details are set forth. However, it is understood that embodiments of the invention may be practiced without these specific details. In other instances, well-known methods, structures and techniques have not been shown in detail in order not to obscure an understanding of this description.

Thus, while there has been described what are believed to be the preferred embodiments of the invention, those skilled in the art will recognize that other and further modifications may be made thereto without departing from the spirit of the invention, and it is intended to claim all such changes and modifications as falling within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations and implementations are possible within the scope of the disclosure. Accordingly, the disclosure is not to be restricted except in light of the attached claims and their equivalents.

What is claimed is:

1. A computer-implemented method comprising:
collecting, using one or more processors, a plurality of data from one or more sources;
selecting, using the one or more processors and based on the plurality of data, one or more procedures and/or conditions upon which performance evaluation of one or more entities is based;
selecting, using the one or more processors and based on the plurality of data, one or more entities for which the performance evaluation is conducted;
for each of one or more procedures and/or conditions:
determining, using the one or more processors and using one or more models, a performance score for each of the one or more selected entities based on one or more performance indicators, wherein the one or more performance indicators include one or more of:
one or more risk-adjusted outcomes,
one or more process measures, and
one or more structural measures; and
generating, using the one or more processors, a rank for each of the one or more selected entities based on the performance score determined for each of the one or more selected entities; and
causing, using the one or more processors, a display of the rank for the one or more selected entities in association with the one or more procedures and/or conditions in a user interface of a device.

2. The computer-implemented method of claim 1, wherein the one or more procedures and/or conditions are selected based on one or more of: a frequency of admission, an ability to make entity-to-entity comparisons, or a presence of a sufficient degree of risk or complexity such that a quality of an entity's performance is important.

1. The computer-implemented method of claim 1, wherein the one or more procedures and/or conditions are selected based on an inclusion criteria and/or an exclusion criteria.

2. The computer-implemented method of claim 3, wherein the inclusion criteria and/or the exclusion criteria are defined based on one or more of maximal homogeneity, maximal sample size, or minimal coding variation.

3. The computer-implemented method of claim 1, wherein the one or more entities are selected by excluding, from a plurality of entities associated with a database, each entity that is associated with one or more attributes indicative of non-inclusion.

4. The computer-implemented method of claim 5, wherein the one or more attributes include one or more of:
federal government ownership;
an absence of Medicare provider number;
an absence of clinically-integrated facility;
a primary service (SERV) code indicating a service type other than a specific set of conditions; or
a volume insufficient to allow estimation for at least one outcome.

5. The computer-implemented method of claim 1, wherein the one or more models are selected by:
evaluating model statistics for combinations of performance indicators; and
determining the one or more models associated with an optimal combination of one or more of: a number of performance indicators, a model fit, or consistency with models in related cohorts.

6. The computer-implemented method of claim 1, wherein the one or more risk-adjusted outcomes have been risk-adjusted using a multi-level logistic regression model.

7. The computer-implemented method of claim 1, wherein the one or more risk-adjusted outcomes have been risk-adjusted based on one or more risk-adjustment variables, wherein the one or more risk-adjustment variables include one or more:
age at admission;
inbound transfer status;
year of hospital admission;
Elixhauser comorbidities;
Medicare status code;
socioeconomic status;
condition cohort-specific covariates;
surgical cohort-specific covariates;
history of stroke; or
Covid-19 diagnosis.

8. The computer-implemented method of claim 1, wherein the one or more risk-adjusted outcomes include one or more of:
mortality within a pre-determined time period;
unplanned readmission within a pre-determined time period;
surgical site infection, hip replacement, knee replacement, Abdominal Aortic Aneurysm Repair (AAA), Heart Bypass Surgery (CABG), and Aortic Valve Surgery (AVR) cohorts;
revision within a pre-determined time period, hip replacement, and knee replacement cohorts;
prolonged hospitalization, leukemia, lymphoma and myeloma and procedure cohorts;
discharge to a location other than a patient's home;
stroke on procedure date, CABG, AVR, and Transcatheter Aortic Valve Replacement (TAVR) cohorts; or
time spent at home within a pre-determined time period of discharge.

9. The computer-implemented method of claim 1, wherein the one or more process measures include one or more of:
worker flu immunization;
noninvasive ventilation;
patient experience;
board certification;
emergency room visits after chemotherapy;
unplanned visits after colonoscopy;
compliance with a septic shock bundle; or
public transparency.

10. The computer-implemented method of claim 1, wherein the one or more structural measures include one or more of:
volume of procedures;
nurse staffing; or
National Cancer Institute (NCI)-designated Cancer Center and/or American College of Surgeons (ACS) Commission on Cancer.

13. The computer-implemented method of claim 1, wherein the one or more sources include one or more of:
publicly available indicators;
Medicare Beneficiary Summary Files (MBSF);
Medicare inpatient Limited Data Set Standard Analytical Files (LDS SAF);
Medicare outpatient limited data set standard analytical files;
Medicare Skilled Nursing Facility (SNF) limited data set standard analytical files;
American Hospital Association (AHA) annual survey;
Hospital Consumer Assessment of Healthcare Providers and Systems Survey (HCAHPS);
Orthopedic Board Certification Data; or
total volume data from American Hospital Directory (AHD).

14. A system comprising:
one or more processors of a computing system; and
at least one non-transitory computer readable medium storing instructions which, when executed by the one or more processors, cause the one or more processors to perform operations comprising:
collecting a plurality of data from one or more sources;
selecting, based on the plurality of data, one or more procedures and/or conditions upon which performance evaluation of one or more entities is based;
selecting, based on the plurality of data, one or more entities for which the performance evaluation is conducted;
for each of one or more procedures and/or conditions:
determining, using one or more models, a performance score for each of the one or more selected entities based on one or more performance indicators, wherein the one or more performance indicators include one or more of:
one or more risk-adjusted outcomes,
one or more process measures, and
one or more structural measures; and
generating a rank for each of the one or more selected entities based on the performance score determined for each of the one or more selected entities; and
causing a display of the rank for the one or more selected entities in association with the one or more procedures and/or conditions in a user interface of a device.

15. The system of claim 14, wherein the one or more procedures and/or conditions are selected based on one or more of: a frequency of admission, an ability to make entity-to-entity comparisons, or a presence of a sufficient degree of risk or complexity such that a quality of an entity's performance is important.

16. The system of claim 14, wherein the one or more procedures and/or conditions are selected based on an inclusion criteria and/or an exclusion criteria.

17. The system of claim 16, wherein the inclusion criteria and/or the exclusion criteria are defined based on one or more of maximal homogeneity, maximal sample size, or minimal coding variation.

18. The system of claim 14, wherein the one or more entities are selected by excluding, from a plurality of entities associated with a database, each entity that is associated with one or more attributes indicative of non-inclusion.

19. The system of claim 18, wherein the one or more attributes include one or more of:
federal government ownership;
an absence of Medicare provider number;
an absence of clinically-integrated facility;
a primary service (SERV) code indicating a service type other than a specific set of conditions; or
a volume insufficient to allow estimation for at least one outcome.

20. A non-transitory computer readable medium, the non-transitory computer readable medium storing instructions which, when executed by one or more processors of a computing system, cause the one or more processors to perform operations comprising:
collecting a plurality of data from one or more sources;
selecting, based on the plurality of data, one or more procedures and/or conditions upon which performance evaluation of one or more entities is based;
selecting, based on the plurality of data, one or more entities for which the performance evaluation is conducted;
for each of one or more procedures and/or conditions:
determining, using one or more models, a performance score for each of the one or more selected entities based on one or more performance indicators, wherein the one or more performance indicators include one or more of:
one or more risk-adjusted outcomes,
one or more process measures, and
one or more structural measures; and
generating a rank for each of the one or more selected entities based on the performance score determined for each of the one or more selected entities; and
causing a display of the rank for the one or more selected entities in association with the one or more procedures and/or conditions in a user interface of a device.
